(19) United States
(12) Patent Application Publication
(10) Pub. No.: US 2019/0144368 A1
(43) Pub. Date: May 16, 2019

(54) Compounds and compositions for biofilm prevention

(71) Applicant: INHIBIO AS, Oslo (NO)

(72) Inventors: Lene Karine VESTBY, Oslo (NO); Live Lingaas NESSE, Østerås (NO)

(21) Appl. No.: 16/315,799

(22) PCT Filed: Jul. 5, 2017
(86) PCT No.: PCT/NO2017/050181

§ 371 Date: Jan. 7, 2019

(30) Foreign Application Priority Data
Jul. 8, 2016 (NO) 20161136

(51) Int. Cl. C07C 49/297; C07C 49/303; C07C 49/307; A01N 35/06; A01N 25/04

(52) U.S. Cl. CPC C07C 49/297; C07C 49/303; A61K 31/122; A01N 35/06; A01N 25/04; C07C 49/307

(57) ABSTRACT

The present invention relates to compounds and compositions for effective biofilm prevention. Particularly, the invention provides compositions comprising certain cyclic ketones found to be efficient in preventing bacterial biofilm formation.

### FIELD OF THE INVENTION

The present invention relates to compounds and compositions for effective biofilm prevention. Particularly, the invention provides compositions comprising cyclic ketones found to be efficient to prevent bacterial biofilm build-up.

### BACKGROUND OF THE INVENTION

Biofilm is defined as microbially derived sessile communities characterized by cells that are attached to a substratum or interface or to each other, are embedded in a matrix of extracellular polymeric substances that they have produced, and exhibit an altered phenotype with respect to growth rate and gene transcription. Biofilms are almost universal in both natural and manmade environments, and they can develop on innumerable surface types including polymers, glass, stainless steel, water pipes, implants, wounds and teeth. In a biofilm, it has been shown that bacteria are 20 to 1000 times less sensitive to biocides, like e.g. disinfectants and antibiotics compared to planktonic bacteria. For this reason, once microorganisms are established in biofilm, they are very difficult to eradicate.

In the marine environment, biofilms constitute a costly problem both for the maritime industry, for aquaculture and for the petroleum industry, to mention a few. For the maritime industry, biofouling (the colonization of submerged surfaces by unwanted organisms such as bacteria, barnacles and algae) has detrimental effects on shipping and leisure vessels, as well as aquaculture and other marine installations. Biofilm formation by bacteria and other microorganisms serves as the foundation for biofouling. Once a biofilm forms, it is easier for other marine organisms such as barnacles to attach. Fouling on hulls of ships increases the frictional drag and can reduce speed in excess of 10%. A vessel with a fouled hull can burn as much as 40% more fuel, which has an impact on fuel costs and on additional greenhouse gas production (estimated to be 20 million tons per annum). In some instances, the hull structure and propulsion systems can become damaged. Fouled hulls are also involved in the spread of ‘alien species’ around the world, potentially threatening the balance of sensitive ecosystems.

There is an increased focus in the oil and gas industry on the control of microorganisms. Water injection is by far the preferred method of increasing oil recovery efficiency. It also introduces nutrients such as sulphates which stimulate microbial growth in reservoirs and in production facilities. Increases in the production of hot water rich in nutrients lead to three major problems:
Corrosion, which increases the risk of equipment failure and leads to higher operating costs. A major part of this is due to microorganisms.
The production of the poisonous gas, such as hydrogen sulphide (H₂S), in reservoirs—also called reservoir souring. This will result in increased corrosion, and the problem increases in the use of production chemicals (scavengers). It also increases the risks to personnel onboard.
Biofouling, which reduces the efficiency of production equipment such as heat exchangers.

Biofilms cause complications in several industrial sectors. These vary from the oil and gas industry, animal feed production plants, human and animal medical equipment industry to the marine and maritime production industry, among several others. For instance, in the aquaculture, where biofilms cause problems as these grow on the fish nets and cause increased costs for cleaning the nets. Also, in the fish processing industry, the biofilm can harbor pathogenic bacteria like Listeria monocytogenes, which is a major food safety threat. It can cause severe foodborne disease (listeriosis) with high hospitalization rates and mortality rates in excess of 30% in humans, making safe food handling paramount to ensure public health. The bacterium is ubiquitous in the environment and is regularly found to contaminate food processing plants, increasing the risk of cross-contaminated products. In particular, the safety of consuming ready-to-eat products like vacuum-packed smoked fish has drawn scrutiny from the public and the scientific communities, as these products have been associated with listeriosis outbreaks. Unlike many other foodborne bacteria, Listeria tolerates salty environments and can even multiply at temperatures as low as 2-4° C. Despite comprehensive surveillance of Listeria in the facilities and products, as well as rigorous cleaning and disinfection routines, Listeria remains a battle. Results from a recent research project financed by the Norwegian Seafood Research Fund (FHF) showed that Listeria is very difficult to eradicate, as more than 30% of samples from production facilities still were positive for Listeria after disinfection. Furthermore, the data indicated that the participating plants might harbor “house strains”, i.e. strains that are able to persist in the facilities for an extended period of time.

As seen from the examples above, there are numerous areas where the development of bacterial biofilm causes large problems, such as in aquaculture and food production environments. Further, biofilms are found virtually everywhere and is potentially a problem in practically all terrestrial and marine environments. The available products on the market for the treatment of bacterial biofilms, such as biocides, aim at killing bacteria and removing already established biofilms. Some of these measures might work for certain bacteria, while leaving others less affected, and may even promote biofilm formation. Bacteria organized in biofilms are protected and robust, thus the treatment is challenging. Currently, mechanical removal, use of biocides and desiccation are widely used antibacterial and anti-biofilm measures. Bacteria can develop resistance to biocides, enabling them to grow, divide and pass on their resistance to their descendent. The emergence of bacterial resistance to biocides and the possible linkage between biocide and antibiotic resistance is a major topic of discussion and concern. Further, biocides are chemical agents that are usually toxic, not only for the end user, but also for the environment. The toxicity of some biocides has been particularly well described, e.g, the high-level disinfectant glutaraldehyde, the use of which has been associated with dermatitis and occupational asthma. Hence, there is a need for new methods and compositions for preventing bacterial biofilm to build up.

Some biofilm inhibitors have been suggested in the prior art. By way of example JP2007/091706 discloses compounds from cearwood oil and palmarosa oil, suggesting that caryophyllene, cedrene, cedrol, cedryl acetate, vetiverol, nerolidol, santa roll, phytol, linalool, geraniol, limonene, 1-carvone, menthone, menthol, cis-jasmone, dihydrojasmone and dihydromethyl jasmonate may be used as biofilm suppressing agents. The compounds suggested are of different chemical groups, all being extracts from plants and they have previous been used typically in perfumes or as aroma or fragrance additives. Further, WO09/074792 is directed to alkyl- or halosubstituted benzoquinones or hydroquinones for use in treatment of a periodontal disease, e.g. based on a bacterial infection or a bacterial composition of plaque.

Alternative compounds have now been found which are efficient in preventing bacterial biofilm formation. It has now surprisingly been found, that certain cyclic ketones are efficient to prevent bacterial biofilm formation or build-up. A great advantage of the compounds of the claimed compositions is that they prevent or decrease the build-up of biofilm without killing the bacteria. They will, therefore, delay or prevent the formation of bacterial biofilm as well as ease their eradication. They will act in combination with, and increase the effect of, e.g. anti-fouling agents and biocides. Further, the compounds can be applied to or incorporated into a variety of materials and compositions, eg. steel, polymers, and other substances as well as mixed in solutions and paints among others, to inhibit and prevent biofilm formation.

### BRIEF SUMMARY OF THE INVENTION

The invention relates to a composition comprising at least one cyclic ketone as disclosed below, or derivatives thereof, in an amount which prevents biofilm formation. Hence, the invention relates to a solution, matrix, powder, gel or coating comprising one or more cyclic ketones or derivatives thereof at a concentration of preferably 0.05-500 mg/ml or 0.1-300 mg/ml or at least used in a concentration of 1-100 mg/ml to prevent biofilm formation.

### DETAILED DESCRIPTION OF THE INVENTION

The invention describes a biofilm prevention technology typically comprising use of molecules having the general structure 1 or 2 alone, or in combination using two or more different molecules.

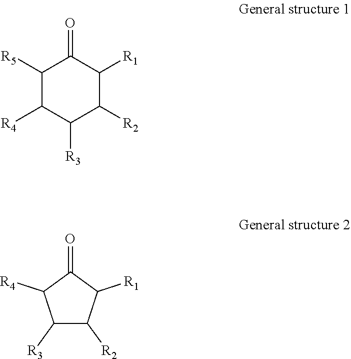

R¹, R², R³, R⁴ and R⁵ are independently selected from the group consisting of hydrogen, oxygen, hydroxy, methoxy, ethoxy, methyl and ethyl.

The invention relates to a solution, matrix, powder, gel or coating comprising one or more cyclic ketones or derivatives thereof at a concentration of preferably 0.05-500 mg/ml or 0.1-300 mg/ml or at least used in a concentration of 1-100 mg/ml to prevent biofilm formation.

Preferably the solution, matrix, powder, gel or coating according to the invention comprises cyclic ketones with 5-7 carbon atoms in the cyclic structure or derivatives thereof at a concentration of between 0.05 and 500 mg/ml to prevent biofilm formation.

More preferable the solution, matrix, powder, gel or coating composition according to the claims comprises one or more of the compounds of General structure 3:

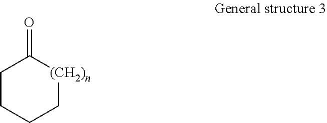

wherein n is 0-2; optionally substituted by 1-3 groups selected from ═O, —OH, —C₁₋₆ alkyl, —O—C₁₋₆ alkyl, —CH═O and —C(OH)═O;
or salts, hydrates, solvates or tautomeres thereof;
at a concentration of between 0.05 and 500 mg/ml or mg/g to prevent biofilm formation.

Accordingly, in one aspect the invention provides a composition comprising a compound of General structure 3,

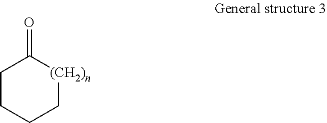

wherein
n is 0-2;
optionally substituted by 1-3 groups selected from ═O, —OH, —C₁₋₆ alkyl, —O—C₁₋₆ alkyl, —CH═O and —C(OH)═O;
or salts, hydrates, solvates or tautomeres thereof;
   in an amount preventing biofilm formation.

The optional groups which substitute the cycle are called R-groups. In a preferred embodiment, when either of the R-groups are a —C₁₋₆ alkyl group these are preferably selected from the group of methyl and ethyl. Hence, the 1-3 R-groups are preferably independently selected from the group of carbonyl, H, —OH, methyl and ethyl, —O—C₁₋₆ alkyl, —CH═O and —C(OH)═O.

In one embodiment, when n is 1 and neither of the R-groups are a carbonyl, neither of the R-groups are iso-propyl. Particularly, 2-isopropyl-5-methylcyclohexane-1-on (Menthone) is disclaimed from the group of compounds of the composition according to the invention.

In one embodiment, at least one of the R-groups is a carbonyl group. In a preferred embodiment, the compound of the composition is a cyclic diketone, and more preferably the compound is a 1,3-cyclopentadion, a 1,3- or 1,4-cyclohexanedione, or a 1,3- or 1,4-cycloheptadione, optionally substituted, i.e. optionally substituted with the R-groups given for General structure 3 above, and e.g. substituted with either of one or more methyl or ethyl groups.

Even more preferable the composition is a solution, matrix, powder, gel or coating comprising compounds selected from the group consisting of:
1,3-cyclohexanedione;
1,4-cyclohexanedione;
Cyclopentanone;
5-ethylcyclohexane-1,3-dione; and
5-methyl-1,3-cyclohexanedione.

In one embodiment, at least one R-group is different from hydrogen.

In a preferred embodiment, the compound of the composition is selected from the group of 1,3-cyclohexanedione, 1,4-cyclohexanedione, 5-ethylcyclohexane-1,3-dione and 5-methyl-1,3 cyclohexanedione.

In one embodiment, the compounds of the composition are able to undergo keto-enol tautomerism. Particularly 1,3-cyclodions, such as 1,3-cyclohexanediones, particularly such diones wherein neither or maximum one substituents are present in the C-2 position, have been found favourable as they can undergo keto-enol tautomerism. NMR-data of cyclohexane-1,3-dione support that in aqueous solution this compound exists in a highly enolised form where electrons are delocalised between the C-1-oxygen/C-1, C-2 and C-3/C-3-oxygen thereby rendering the C-2 methylene protons relatively acidic. Further, it is shown that tautomers of cyclohexane-1,3-dione are stable at least over one day and that higher pH favours the observed enol tautomer. It is believed that the acidity of the C-2 protons affect the solubility of the compound, providing a higher solubility than e.g. similar 1,4-carbocyclic compounds. E.g. as 1,3-cyclohexanedione is generally more potent than 1,4-variant it is plausible that the keto-enol tautomerism property is the reason for this.

The cyclic ketone compounds of the compositions are chemically synthesized, and are preferably not e.g. obtained from plant oils or extracts. Higher purity and ease of production are clear benefits compared to plant extraction. Even though some plant extracts may have some effect on reducing biofilm formation, some oil extracts, such as e.g. extracts comprising menthone, actually has a promoting effect on bacteria growth.

The compounds of the invention have also shown to be stable when incorporated into different matrices, and have a dose dependent action. When compared to other biofilm inhibitors, such as menthone, certain differences were noted. For instance, in addition to a superior effect at a concentration of 1 mg/ml the claimed compounds inhibit even more biofilm formation at higher concentrations, such as at 10 mg/ml. In comparison, menthone has a decreased effect at 10 mg/ml, which is a commonly seen effect of plant-based compounds. Reference is made to Example 1, wherein the biofilm production in presence of 1,3-cyclohexanedione and methone is compared, and to the results shown in FIG. 11. Further, on in silico studies using Epi Suite v.4.1 the compounds were shown to be degradable within days-weeks in addition to be readily biodegradable on the ready biodegradability prediction. Reference is made to Example 5. On the contrary, when a similar estimation was performed on menthone this compound was shown to be not readily biodegradable. This proves that in addition to be non-toxic effective alternatives to prevent biofilm build-up the compounds of the invention also have a minimal impact on the environment compared to commonly used biocides and other anti-biofilm compounds.

Further, as clearly seen from the formulas above, the compounds of the composition are not aromatic and are not heterocycles.

The compounds of the compositions have a favourable toxicological profile and are generally less toxic than biocides. They are biogradable and it is not likely that microorganisms will develop resistance against the compounds disclosed. Hence, they are not bio-accumulating.

The disclosed compounds are efficient and potent at various physical conditions. In one embodiment, the compositions will typically be potent at a temperature range of 2-100° C., such as 4-90° C., and at a pH range of 2-9, and at pressures down to about 300 bar, although the best effect is seen at normal conditions. In one embodiment, it is for seen that compositions of the invention may be used in subsea systems, wherein sea temperature may be as low as 4° C. and where they must withstand the high pressure at depths e.g. of 3000 meter (i.e. 300 bars).

The composition, i.e. the solution, matrix, powder, gel or coating according to any of the claims can be combined with one or more biocides and/or antibacterial agents to prevent biofilm formation. Hence, biocides and/or antibacterial agents may either be included in the compositions of the invention, or may be used or applied separately.

Preferably the solution, matrix, powder, gel or coating is combined with biocides/or antibacterial agent selected from disinfectants and general biocidal products, preservatives, pest control agents or other biocidal products like antifouling agents.

The solution, matrix, powder, gel or coating above is preferably combined with biocides/or antibacterial agent selected from the group consisting of 4-hydroxy-3-methoxybenzaldehyde, cetylpyridinium chloride, quorum sensing inhibitors, biguanides, iodophors, quaternary ammonium compounds, Boric acid, cationic tensides, alcohol based, chlorine based, peroxy based and acid based compounds, tetracyclines, Amphenicols, Beta-lactam antibiotics, Sulphonamides and trimetophrim, macrolides, linkosamides and streptogramins, Aminoglycosides, Quinolones and other antibacterial compounds.

The solution, matrix, powder, gel or coating composition of the invention can be used in a two-step process to prevent biofilm formation. E.g. it can be used after the initial treatment with a biocide, antiseptic or antibiotic as a prevention of further biofilm build-up.

Typically the solution, matrix, powder, gel or coating composition according to any of the claims of the invention comprises the compound of the formulas above, in addition to ingredients and additives, making it applicable for a range of uses as:
a. A solvent or solvent mixture;
b. An industrial paint or varnish;
c. Anti-fouling coatings and/or impregnations for marine use;
d. Anti-fouling coatings for maritime use;
e. A coating bound to the surface of or mixed in polymer material;
f. Solutions or coatings attached/linked to inert surfaces;
g. A coating bound to the surface of or mixed in fiber glass materials.
h. A solution, ointment, or dressing for use in human or veterinary medicine or for a medical purpose.

The compositions of the invention comprise molecules selected from the general structures above. The molecules are known molecules not previously applied or used as biofilm inhibitors. The claimed invention is directed to a biofilm prevention technology/anti-biofilm technology, as the technology does not remove biofilm or kill bacteria as biocides do. The disclosed compounds strictly prevent the bacteria from establishing and forming biofilm, leaving the bacteria ‘free floating’. The mechanism of action is still being explored, but one theory is that the group of cyclic ketones affect how the microorganisms communicate with each other.

The following are examples of uses of the claimed compositions exemplified in the application or substantiated based on literature.
1. One or more of the disclosed molecules selected from general structure used in a solvent to a concentration ranging from preferably 0.05 to 500 mg/ml, or 0.1-300 mg/ml or at least used in a concentration of 1-100 mg/ml to prevent biofilm formation.
2. One or more of the disclosed molecules selected from general structure mixed in industrial paint and varnishes to a concentration ranging from 0.05 to 500 mg/ml to prevent biofilm formation.
3. One or more of the disclosed molecules selected from general structure mixed in anti-fouling coatings and or impregnations for marine use to a concentration ranging from 0.05 to 500 mg/ml to prevent biofilm formation.
4. One or more of the disclosed molecules selected from general structure mixed in anti-fouling coatings and or impregnations to be used as an anti-slime technology at a concentration ranging from 0.05 to 500 mg/ml to prevent biofilm formation.
5. One or more of the disclosed molecules selected from general structure mixed in anti-fouling coatings for maritime use to a concentration ranging from 0.05 to 500 mg/ml to prevent biofilm formation.
6. One or more of the disclosed molecules selected from general structure mixed in or attached to polymer materials to a concentration ranging from 0.5 to 500 mg/ml to prevent biofilm formation.
7. One or more of the disclosed molecules selected from general structure chemically and/or physically attached to inert surfaces (such as metal, glass and so on) to a concentration ranging from 0.05 to 500 mg/ml to prevent biofilm formation.
8. One or more of the disclosed molecules selected from general structure bound to the surface of or mixed in fibre glass materials to a concentration ranging from 0.5 to 500 mg/ml to prevent biofilm formation.
9. One or more of the disclosed molecules selected from general structure in a concentration ranging from 0.05 to 500 mg/ml to prevent biofilm formation intended for use in human or animal medicine. Comprising use on implants, wound dressings, wound treatment products, suture materials, ointments, spray, jelly or any other product used in animal or human medicine. Hence, in one embodiment the invention provides a composition for use in human medicine, such as for use as a medicament, comprising a compound according to any of the formulas above. Further, the invention provides a composition for use in treatment, comprising a compound according to any of the formulas above.
10. One or more of the disclosed molecules selected from general structure in a concentration ranging from 0.05 to 500 mg/ml to be used to potentiate the effect of biocides such as disinfectants, surfactants, antibacterial, electrolyzed water, anti-biofilm specific compounds or antiseptic solutions to prevent biofilm formation. Where the biocides are used in a concentration according to the manufacturer's recommendation or lower.
11. One or more of the disclosed molecules selected from the general structure in a concentration ranging from 0.05 to 500 mg/ml in combination with biocides such as disinfectants, surfactants, antibacterial, electrolyzed water, anti-biofilm specific compounds or antiseptic solutions to prevent biofilm formation. Where the biocides are used in a concentration according to the manufacturer's recommendation or lower.
12. One or more of the disclosed molecules selected from the general structure in a concentration ranging from 0.05 to 500 mg/ml according to number 9-11 where the disinfectants are intended for any of the following groups: Humans and animals, Veterinary hygiene, Food and feed area, preservatives for products during storage and drinking water. Comprising: Air disinfectants, aldehydes, alcohols, oxidizing agents, silver, phenolics, quaternary ammonium compounds, Biguanide, Thymol based disinfectants, ultraviolet germicidal radiation, Sodium bicarbonate and lactic acid among others.
13. One or more of the disclosed molecules selected from the general structure in a composition with a concentration ranging from 0.05 to 500 mg/ml according to number 9 and 10, comprising a surfactant selected from any of the following groups: Anionic, cationic, zwitterionic, non-ionic, ionic surfactants and bio surfactants among others.
14. One or more of the disclosed molecules selected from the general structure in a composition with a concentration ranging from 0.05 to 500 mg/ml according to 9 and 10, comprising an antiseptics selected from any of the following groups: Alcohols, quaternary ammonium compounds, boric acid, brilliant green, chlorhexidine gluconate, hydrogen peroxide, iodine, Manuka honey, Mercurochrome, Octenidine dihydrochloride, Phenol, Sodium Chloride, polyhexanide, Sodium hypochloride, Calcium hypochloride, Sodium bicarbonate or Balsam of Peru among others.
15. One or more of the disclosed molecules selected from the general structure in a composition with a concentration ranging from 0.05 to 500 mg/ml according to number 9 and 10, comprising antimicrobials selected from any of the following groups comprising: Tetracyclines, Aminophenichols, β-lactam antibiotics (penicillins and others), Sulphonamides and thrimetophrim, Macrolides, lincosamides and Streptogramines, Aminoglycosides, Quinolones, Vancomycin, Teicoplanine, Colistin, Fucidinic acid, Metronidazol, Nitrofurantoin, Metenamine, Linezolid and Daptomycine among others.
16. One or more of the disclosed molecules selected from the general structure in a composition with a concentration ranging from 0.05 to 500 mg/ml according to number 9 and 10 comprising: Preservatives such as preservatives for products during storage, film preservatives, wood preservatives, fibre, leather, rubber and polymerized materials preservatives, construction material preservatives, preservatives for liquid-cooling and processing systems, slimicides or working or cutting metal, glass and other materials.
17. One or more of the disclosed molecules selected from the general structure in a composition with a concentration ranging from 0.05 to 500 mg/ml according to number 9 and 10 comprising biocides in antifouling products.
18. One or more of the disclosed molecules selected from the general structure in a composition with a concentration ranging from 0.05 to 500 mg/ml together with and comprising groups of adjuvants and additives such as emulsifying agents, alkylating agents, oxidizing agent, wetting agents, humectants, surface active agents, chelating agents and buffering agents, among others.
19. One or more of the disclosed molecules selected from the general structure in a compositions with a concentration ranging from 0.05 to 500 mg/ml together with agents to enhance or mask smell and taste, where the agents are selected from any of the following groups, as examples consisting of natural or artificial: lime, lemon, orange, pineapple, grapefruit anisaldehyde, vanillin and its derivatives, red chilipepper, anethole, dihydroanethole, eugenol, ethyl maltol and mixtures thereof, benzaldehyde and its derivatives, cinnamon, clove, bay, allspice, anise, wintergreen, spearmint, peppermint, cherry and mixtures thereof.
20. One or more of the disclosed molecules selected from the general structure in a composition in a concentration ranging from 0.05 to 500 mg/ml together with agents to enhance or mask colour with the following examples, being natural or artificial: Brilliant blue FC, Indigotine, Fast green FCF, Erythrocine, Allura red, Vanillic acid, Ethyl-vanillin, O-vanillin, turmeric, saffron, anisaldehyde, Tartrazine, Sunset yellow or titanium oxider.

The disclosed compounds are unique as anti-biofilm agents in the fact that they have a non-toxic reduction of the establishment of biofilm and can be incorporated in different compositions, materials (polymer, fiber glass, textiles etc.) and solutions (paint, varnish, anti-fouling etc.). They can be used solely in solution (such as paint, coatings) as well as in combination with biocides, antiseptics and antibiotics to further enhance the effect of these. When used together with other active ingredients they can be used in combination with or as part of a two-step process. In the latter case they can be used after the initial treatment with a biocide, antiseptic or antibiotic as a prevention of further biofilm build-up. All but one (5-methyl-1,3-cyclohexanedione 98% at 1 mg/ml) of the biofilm formation results show a statistically significant decrease in biofilm formation. This is shown using Confidence interval (CI) in the statistical calculation (CI not including 100% are considered statistically significant at p<0.05).

In another aspect, the invention provides compositions as disclosed above, for use in preventing biofilm formation. Further, in another aspect, the invention provides a method for preventing biofilm formation, including the use of a composition as disclosed above. These further aspects include the same elements and embodiments as disclosed for the composition.

### LIST OF FIGURES

In the Figures the Y-axis provides the % biofilm formation.

FIG. 1: 1,3-cyclohexanedione. Gram positive and Gram negative bacteria.

The graph shows a decrease in biofilm formation of 74 and 70% produced by Gram positive and Gram negative bacteria respectively, when using 1,3-cyclohexanedione in a concentration of 1 mg/ml. Further, a decrease of 98% was seen at a concentration of 10 mg/ml in Gram positive bacteria and 87% in Gram negative bacteria. The asterisks show that the reduction is statistically significant (p<0.05).

FIG. 2: 1,4-cyclohexanedione. Gram positive and Gram negative bacteria. The graph shows a 53% decrease in biofilm produced by Gram positive bacteria when using 10 mg/ml 1,4-cyclohexanedione. Similarly, a decrease of 91% was seen in biofilm formation by Gram negative bacteria. The asterisks show that the reduction is statistically significant (p<0.05).

FIG. 3: Cyclopentanone. Gram negative bacteria. The graph shows a decrease of 77% in biofilm formation by Gram negative bacteria using cyclopentanone at a concentration of 10 mg/ml. The asterisk shows that the reduction is statistically significant (p<0.05).

FIG. 4: 5-ethylcyclohexane-1,3-dione. Gram positive bacteria. The graph shows a decrease of 55% in biofilm production using 5-ethylcyclohexane-1,3-dione at 1 mg/ml, and 74% decrease at 2 mg/ml and 98% decrease at 10 mg/ml. The asterisks show that the reduction is statistically significant (p<0.05).

FIG. 5: 5-methyl-1,3-cyclohexanedione. Gram positive bacteria. The graph shows a decrease of 32% in biofilm formation using 5-methyl-1,3-cyclohexanedione at 1 mg/ml, 85% at 2 mg/ml and 98% at 10 mg/ml. The asterisks show that the reduction is statistically significant (p<0.05).

FIG. 6: 1,3-cyclohexanedione. Listeria monocytogenes. The graph shows a decrease of 27%, 40%, 53% and 70% using 1,3-cyclohexanedione at 0,625 mg/ml, 2.5 mg/ml, 5 mg/ml and 10 mg/ml respectively. The asterisks show that the reduction is statistically significant (p<0.05).

FIG. 7: 1,4-cyclohexanedione. Sulphate reducing bacteria (SRB). Arrow A in the picture points to the slide with SRB bacterial biofilm included as a control. Arrow B points to a slide that has been immersed in medium containing 1,4-cyclohexanedione at a concentration of 10 mg/ml.

FIG. 8: Cyclopentanone. Sulphate reducing bacteria (SRB). Arrow A in the picture points to the slide with SRB bacterial biofilm included as a control. Arrow B points to a slide that has been immersed in medium containing cyclopentanone at a concentration of 10 mg/ml.

FIG. 9: 1,3-cyclohexanedione. Resistance to increasing temperature. The graph shows biofilm formation after heating the solutions containing 1,3-cyclohexanedione compound to 80° C., 90° C. and 100° C. The graph shows a decrease in biofilm formation of 38%, 40% and 28% respectively, compared to the control (0 mg/ml 1,3-cyclohexanedione). The unheated control solution containing 1,3-cyclohexanedione (1 mg/ml) shows a decrease of 46% in biofilm formation, compared to the sample without the substance added.

FIG. 10: 1,3-cyclohexanedione. Stainless steel. The figure shows the average of log values of the parallel experiments using 1,3-cyclohexanedione in two concentrations. A decrease in biofilm formation was seen with 1,3-cyclohexanedione at a concentration of 1 mg/ml. At 10 mg/ml, we could not detect any colony forming units (CFU's).

FIG. 11: 1,3 Cyclohexanedione and menthone. E. coli. The graph shows a decrease of 46 and 70% in biofilm formation using 1,3-cyclohexanedione at 1 mg/ml, and 97% and 100% at 10 mg/ml. For menthone, the decrease is seen to be 27% and 48% at 1 mg/ml, and 6% and 26% at 10 mg/ml.

### DEFINITIONS

In the present context, the disclosed compounds or molecules are of any of the formulas above, and are typically compounds such as 1,3-cyclohexanedione (C₆H₈,O₂), 1,4-cyclohexanedione (C₆H₁₂O₂), cyclopentanone (C₅H₈O), 5-ethylcyclohexane-1,3-dione (C₈H₁₂O₂) and 5-methyl-1,3 cyclohexanedione (CH₃C₆H₇(═O)₂) for use as anti-biofilm agent in solutions, or incorporated into or onto materials. Examples, but not limited thereto are paint, antifouling coating, polymer, glass or metal surfaces.

When referring to “general structure” any compound falling within either of the formulas shown above are encompassed, such as compounds of general structure 1 and general structure 2, and of General Structure 3.

In the present context, a biofilm is an extracellular matrix community of sessile, stable attached microorganisms, such as bacteria, embedded in a self-produced matrix consisting of various components, including extracellular polymeric substances. Biofilm formation consists of three steps; attachment, growth and detachment in order to recolonize another surface. Extracellular matrix is continuously formed during the first two steps.

In the present context, a biofilm is considered to have been established from the moment when one or more microorganisms is/are irreversibly attached to a surface. Examples, but not limited thereto, of a surface, is a wound and wound area.

The term “surface” is intended to relate to any surface which may be partially or fully covered by a biofilm. Examples, but not limited to, of surfaces are metal, polymer, fibre glass, human skin, epithelial cells, muscle tissue and surgical suture material or any coated or impregnated area.

The term “effective amount” as used herein refers to an amount effect, at dosages and for periods of time necessary to achieve a desired result.

In the present context, for medical use, the term “effective amount” refers to an amount of a compound or compounds that is sufficient to effect treatment when administered to a subject in need of such treatment.

### EXPERIMENTAL

The following examples are illustrations within the scope of the claims.

### Example 1

### Biofilm Formation Experiments

All experiments were performed by using sterile 96-wells polystyrene microtiter plates (Nunc, Nuncleon, Roskilde, Denmark) under conditions promoting biofilm formation by the different bacterial genera. The Inhibio compound preparations of the invention; 1,3-cyclohexanedione, 1,4-cyclohexanedione, cyclopentanone, 5-ethylcyclohexane-1,3-dione and 5-methyl-1,3 cyclohexanedione 98% were solved directly in Tryptic Soy Broth (TSB) 1+1 or Liquid Microbiology Broth (LB) broth without NaCl (LB^{wo}/NaCl) to obtain the test concentrations, i.e. from 1 mg/ml to 10 mg/ml.

### Gram Positive Bacteria

Three Staphylococcus aureus strains of animal origin were used in this study. All strains were stored at −80° C. in Brain Heart Infusion Broth (BHI) (Difco, BD, Franklin Lakes, N.J., USA) supplemented with 15% glycerine (Merck KGaA, Darmstadt, Germany) and were recovered on blood agar at 37.0±1.0° C. The bacterial cultures were then transferred into TSB and were incubated statically overnight at 37.0±1.0° C. to obtain an overnight working culture. A total of 2 μl of this suspension was transferred to each 96 wells of polystyrene microtiter plates (Nunc, Nuncleon, Roskilde, Denmark) containing 198 μl TSB 1+1 with dissolved Inhibio compounds in the following concentrations: 0 mg/ml, 1 mg/ml and 10 mg/ml for 1,3-cyclohexanedione; 0 mg/ml and 10 mg/ml for 1,4-cyclohexanedione; and 0 mg/ml, 1 mg/ml, 2 mg/ml and 10 mg/ml for 5-ethylcyclohexane-1,3-dione and 5-Methyl-1,3-cyclohexanedione in the total amount of broth. Three parallels of each strain were used and the microtiter plates were incubated statically for one day, at 37±1.0° C. After incubation, OD₅₉₅ was measured in a microplate photometer (Multiscan EX; Thermo Fisher Scientific Inc, Waltham, Mass., USA) before the plates were gently washed three times with 290 μl tap water. The plates were dried in room temperature before addition of 220 μl 1% crystal violet (Sigma-Aldrich, St. Louis, Mo., USA). After 30 minutes incubation in room temperature, the plates were washed three times with tap water before the addition of 220 μl ethanol:acetone (70:30 w:w) to dissolve the bound dye. The plates were incubated for 10 minutes in room temperature before OD₅₉₅ was measured after the bound dye was dissolved using ethanol:acetone. For each strain, the result was calculated by subtracting the median OD₅₉₅ of the three parallels of the control (test broth only) from the median OD₅₉₅ of the three parallels of sample. Further, the average result of all three Gram positive strains included in the study were calculated. Three independent experiments were performed and the average was evaluated.

### Gram Negative Bacteria

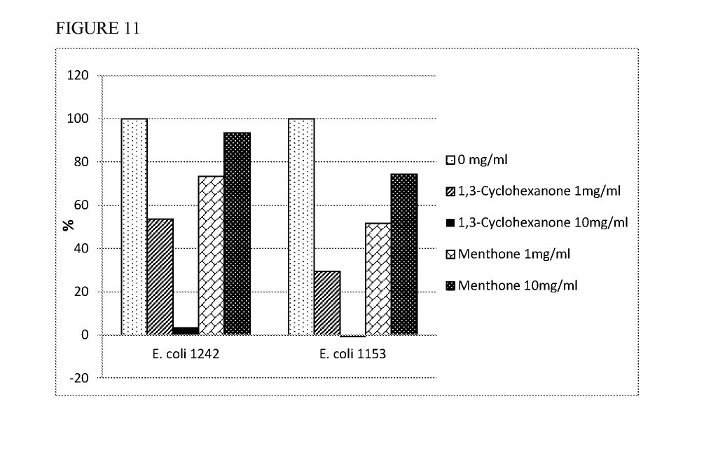

One Salmonella ser. Typhimurium (S. Typhimurium), two Salmonella ser. Agona (S. Agona) and two Escherichia coli (E. coli) isolates were used in these studies. The studies performed using 1,3-cyclohexanedione and 1,4-cyclohexanedione were done on Salmonella isolates only. The studies comparing Menthone to 1,3-cyclohexanedione were using two E. coli strains, E. coli 1242 and E. coli 1153, see FIG. 11. The Salmonella isolates were isolated from Norwegian feed factories except one S. Typhimurium strain which was a culture collection strain (ATCC 14028). The E. coli isolates were of animal origin. All strains were stored at −80° C. in BHI supplemented with 15% glycerine and were recovered on blood agar at 37.0±1.0° C. The bacterial cultures were then transferred into LB broth and incubated statically overnight at 37.0±1.0° C. to obtain an overnight working culture. A total of 30 μl of this suspension was transferred to each well in 96 wells polystyrene microtiter plates (Nunc, Nuncleon, Roskilde, Denmark) in triplets. The wells were containing 100 μl LB^{wo}/NaCl (bacto-tryptone 10 g/l, yeast extract 5 g/l) with 1,3-cyclohexanedione dissolved in concentration 0 mg/ml, 1 mg/ml and 10 mg/ml in the total amount of broth and 1,4-cyclohexanedione and cyclopentanone at 0 mg/ml and 10 mg/ml. The microtiter plates were incubated statically for two days, at 20±1.0° C. After incubation, OD₅₉₅ was measured before the plates were gently washed one time with 150 μl tap water. The plates were dried in room temperature before addition of 140 μl 1% crystal violet (Sigma-Aldrich). After 30 minutes incubation in room temperature, the plates were washed three times with tap water before addition of 140 μl ethanol:acetone (70:30 w:w) and incubated for another 10 minutes in room temperature. OD₅₉₅ was measured in a microplate photometer (Multiscan EX) after the bound dye was dissolved using ethanol:acetone. For each strain, the result was calculated by subtracting the median OD₅₉₅ of the three parallels of the control (test broth only) from the median OD₅₉₅ of the three parallels of sample. Further, the averages of the Gram negative strains were calculated. Three independent experiments were performed and the average was evaluated.

### Results:

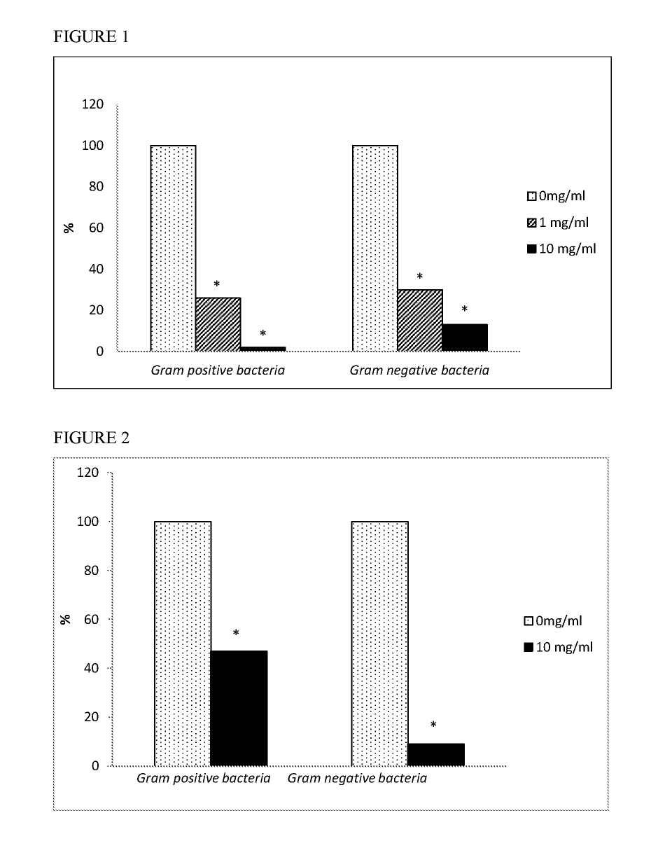

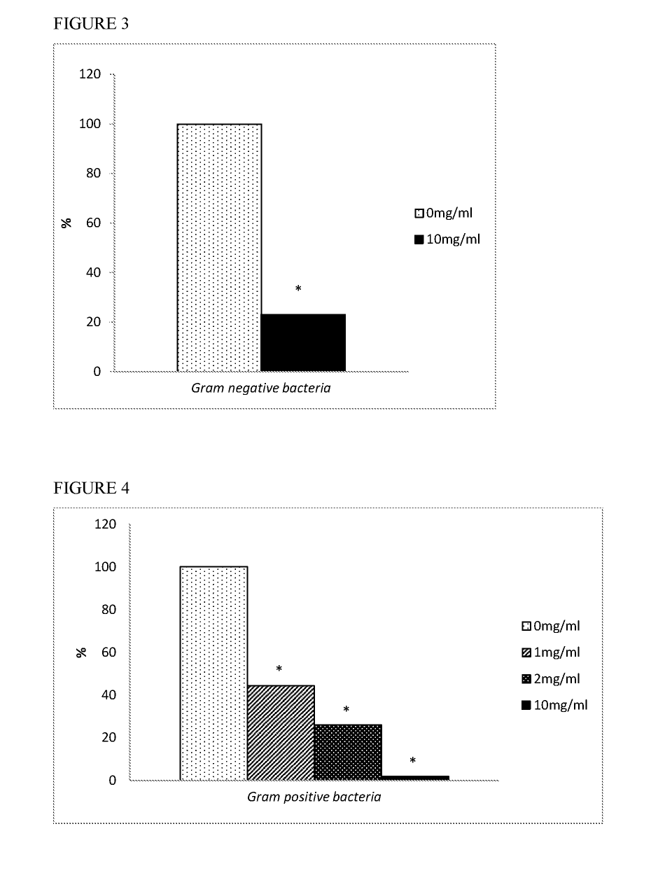

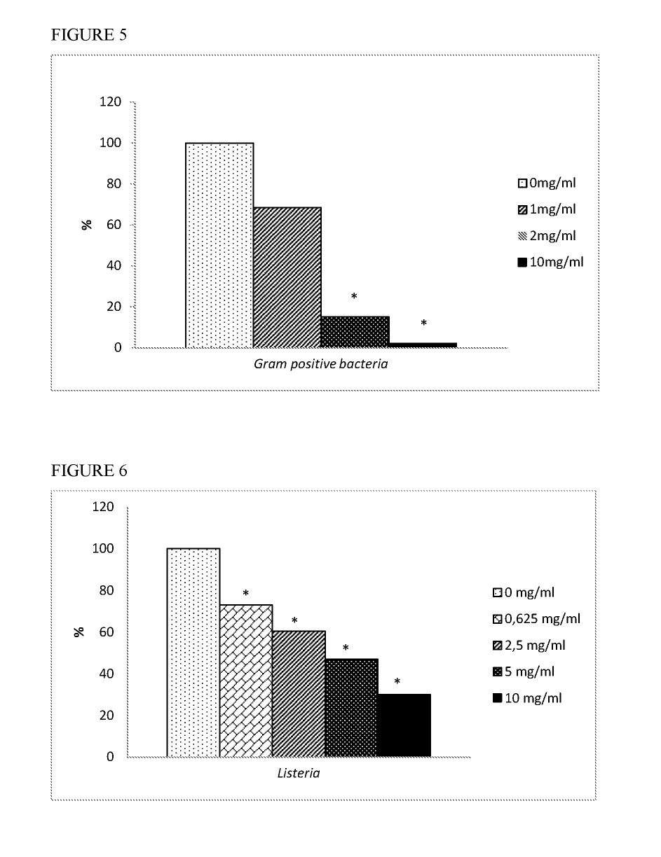

The results are expressed as a decrease in biofilm formation calculated in percentage of the control (without compound of the invention). A decrease of 74 and 70% in biofilm production was found using a concentration of 1 mg/ml 1, 3-cyclohexanedione on biofilm produced by Gram positive and Gram negative bacteria, respectively. Further, a decrease of 98% was seen at a concentration of 10 mg/ml 1,3-cyclohexanedione in Gram positive bacteria and 87% in Gram negative bacteria. For 1,4-cyclohexanedione, the results show a 53% decrease in biofilm produced by Gram positive bacteria at 10 mg/ml. Similarly, a decrease of 91% was seen in biofilm formation by Gram negative bacteria. Using cyclopentanone, a decrease of 77% in biofilm formation by Gram negative bacteria was detected at a concentration of 10 mg/ml. Further, a decrease of 55% of biofilm formation was found using 5-ethylcyclohexane-1,3 dione in 1 mg/ml, 74% in 2 mg/ml and 98% in 10 mg/ml in Gram positive bacteria. Similarly, a decrease of 32% was found using 5-methyl-1,3-cyclohexanedione 98% at 1 mg/ml, 85% decrease at 2 mg/ml and 98% at 10 mg/ml. The studies comparing 1,3-cyclohexanedione and menthone showed a decrease of 46 and 70% using 1,3-cyclohexanedione at a concentration of 1 mg/ml and a decrease of 97 and 100% at 10 mg/ml. On the contrary, menthone showed a decrease of 27 and 48% at 1 mg/ml and at 10 mg/ml the decrease was 6 and 26% (FIG. 11). This shows that menthone fails to show a dose dependent effect, which is a known response by plant based compounds. All results were statistically significant (shown with asterisk) at confidence interval 95% except 5-methyl-1,3-cyclohexanedione 98% at 1 mg/ml. A student's T-test was not performed on the comparative studies with menthone. See FIGS. 1, 2, 3, 4, 5 and 11.

### Listeria

Six strains of Listeria monocytogenes were used in this study isolated from food research. The strains were recovered in 200 μl TSB broth in a microtiter plate and incubated at 37° C. for 24 hours. A total of 5 μl of the bacterial suspension was transferred to a 96 wells polystyrene microtiter plate (Thermo scientific Nucleon Delta surface) together with 200 μl medium (LB^{wo}/NaCl) with 1,3-cyclohexanedione (dissolved to 0,625 mg/ml, 2.5 mg/ml, 5 mg/ml and 10 mg/ml) to each well. Each strain was added in duplex and incubated at 35° C. for 24 hours. After incubation, OD₅₉₅ was measured before the plates were gently washed one time with 200 μl tap water. This was repeated once. The plates were dried in room temperature before addition of 200 μl 0, 1% crystal violet (Sigma-Aldrich). After 30 minutes incubation in room temperature, the plates were washed twice with 200 μl tap water and once with 240 μl. This was followed by the addition of 200 μl ethanol:acetone (70:30 w:w) and incubated for another 10 minutes in room temperature. OD₅₉₅ was measured in a microplate photometer (Multiscan EX) after the bound dye was dissolved using ethanol:acetone. For each strain, the result was calculated by subtracting the average OD₅₉₅ of the two parallels of the control (test broth only) from the average OD₅₉₅ of the two parallels of sample. Three independent experiments were performed. The average of the 6 strains as well as the average between the experiments was calculated.

### Results:

The results from the studies with 1,3-cyclohexanedione are shown as a decrease in biofilm formation in percentage of the control (no claimed compound present). A decrease of 27%, 40%, 53%, 70% was found using 1, 3-cyclohexanedione at 0.625 mg/ml, 2.5 mg/ml, 5 mg/ml and 10 mg/ml respectively. All results were statistically significant at confidence interval 95% (marked with asterisk). See FIG. 6.

### Example 2

### The Effect of 1,4-Cyclohexanedione and Cyclopentanone on Biofilm Produced by SRB (Sulphate Reducing Bacteria) in Fluids.

One SRB isolate (the culture collection strain: ATCC 29579 Desulfovibrio vulgaris subspecies vulgaris) was tested. 1,4-cyclohexanedione and cyclopentanone were diluted in the culture medium ATCC 1249 to a concentration of 10 mg/ml (1%) and added to each their 50 ml Falcon centrifugal tube. A third tube, with 10 ml of medium only (ATCC 1249) was included as a control. 50 μl SRB starting culture was added to each of the three tubes together with a carbon steel coupon. Biofilm was formed on the coupon by incubating at 20° C. for 12 days. After incubation, the coupons were washed in 40 ml sterile saline to remove loosely adhered cells. A sample from this fluid was injected into SRB medium and blackening of the medium was visualized, showing that there were still free-floating bacteria present.

### Results:

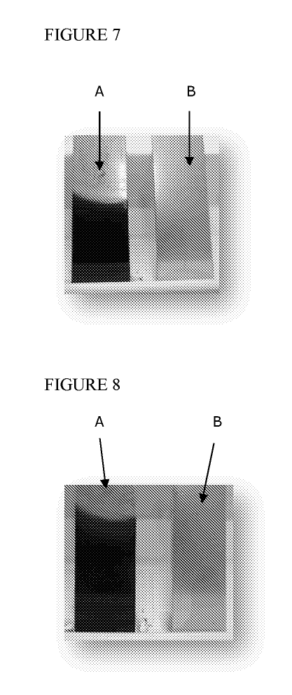

The characteristic black biofilm was seen on the coupon that is not treated. In contrast, on the coupon treated with 1,4-cyclohexanedione or cyclopentanone at a concentration of 10 mg/ml only scarce amount of biofilm could be visualized. See FIGS. 7 and 8.

### Example 3

### The Effect of Increased Temperature on 1,3-Cyclohexanedione

The experiment was performed using 1,3-cyclohexanedione at a concentration of 1 mg/ml. One strain of Salmonella was used in this study. All strains were stored at −80° C. in BHI (Difco, BD, Franklin Lakes, N.J., USA) supplemented 220 μl etanol:aceton 70:30 with 15% glycerine (Merck KGaA, Darmstadt, Germany) and were recovered on blood agar at 37.0±1.0° C. The bacterial culture was transferred into LB broth and was incubated statically overnight at 37.0±1.0° C. to obtain an overnight working culture. 1,3-cyclohexanedione was diluted in LB wo/NaCl at a concentration of 1 mg/ml and divided into 4 tubes. The tubes were heated, for two minutes, to 80° C., 90° C., 100° C. and the last tube was not heated and included as a control. 100 μl from each tube was added to a sterile 96-wells polystyrene microtiter plates (Nunc, Nuncleon, Roskilde, Denmark) together with 30 μl bacterial culture or 30 μl LB^{wo}/NaCl in the case of the blank controls. The plates were incubated for 72 hours at 20° C. The plate was emptied and washed twice using 200 μl tap water in each well each time. This was followed by the addition of 140 μl 1% Crystal violet to each well and, after 30 minutes, the plate was again emptied and washed 3 times using 200 μl tap water. 140 μl etanol:acetone 70:30 was added and OD₅₉₅ was measured after 10 minutes. For each strain, the result was calculated by subtracting the median OD₅₉₅ of the three parallels of the control (test broth only) from the median OD₅₉₅ of the three parallels of sample. At least two experiments were performed and the average was estimated.

### Results:

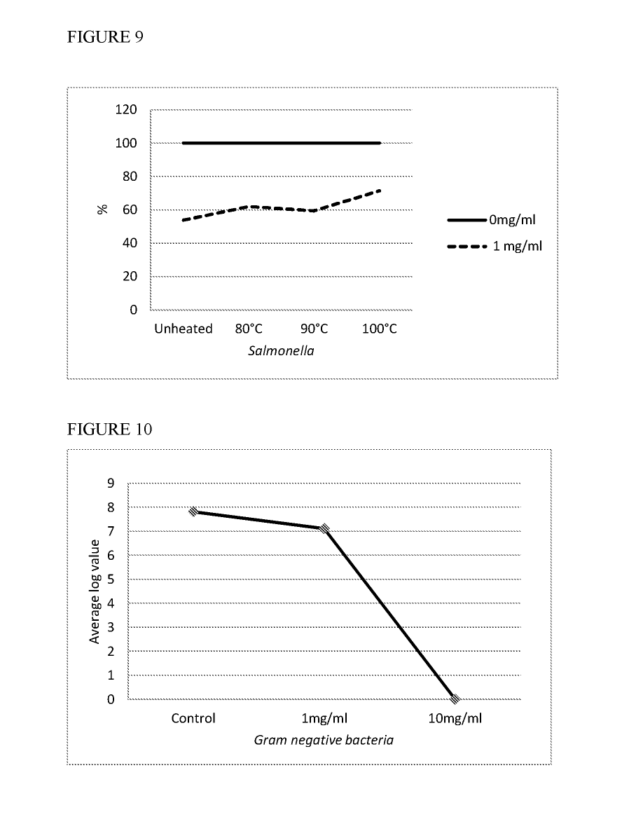

The results show a decrease of 38%, 40% and 28% in biofilm formation after heating the solutions containing 1,3-cyclohexanedione compound to 80° C., 90° C. and 100° C., respectively. The unheated control showed a decrease of 46%. Considering normal variations, this shows that the 1,3-cyclohexanedione compound was still effective in reducing biofilm formation at higher temperatures. See FIG. 9.

### Example 4

### The Effect of 1,3-Cyclohexanedione on Biofilm Formed on Stainless Steel Coupons by Gram Negative Bacteria

One strain of S. Agona (FIG. 10) and one strain of E. coli (FIG. 10) was used in this study. The study was performed using 1,3-cyclohexanedione in a concentration of 1 and 10 mg/ml. The strain was stored at −80° C. in BHI (Difco, BD, Franklin Lakes, N.J., USA) supplemented with 15% glycerine (Merck KGaA, Darmstadt, Germany) and was recovered on blood agar at 37.0±1.0° C. The bacterial culture was then transferred into LB broth and was incubated statically overnight at 37.0±1.0° C. to obtain an overnight working culture. The Inhibio compound was dissolved in LB^{wo}/NaCl to a concentration of 10 mg/ml and then further diluted to a concentration of 1 mg/ml. 10 ml of each solution was added to a 50 ml Falcon tube and, in a third tube, only LB broth^{wo}/NaCl was added as a control. To each tube, 200 μl bacterial culture and an autoclaved stainless steel coupon was added and the tubes were incubated at 20° C. for 72 hours.

Following incubation, each coupon was dipped three times in three different tubes containing physiological saline and further transferred to a tube containing 5 ml cold physiological saline as well as 20 sterile silica glass beads. Each coupon was further scraped with a sterile cell scraper before the coupon was removed and the solution was vortexed at 2000 rpm for one minute. A 10-fold dilution was made in a Nunc microtiterplate (kept on ice) with 180 μl physiological saline and 20 μl of the previous dilution for each well. 100 μl were spread on a blood agar plate and incubated on 37° C. for 24 hours. After incubation, the bacterial colonies were counted. If more than 200 colonies on a plate it was considered overgrown. At least two experiments were performed.

### Results:

The Colony forming units (CFU) on the plate was calculated into CFU in biofilm by multiplying with a factor for each dilution. Further, the log value was calculated of the average of each dilution. A significant decrease was seen with 1,3-cyclohexanedione in a concentration of 1 mg/ml. At 10 mg/ml, there were no bacteria found in the biofilm. See FIG. 10.

### Example 5

### InSilico Studies to Assess Biodegradability

For the compounds 1,3-Cyclohexanone and 1,4-Cyclohexanone InSilico studies were performed using EPIWEB 4.1 and BIOWIN v4.10. The Biowin 3 (the ultimate biodegradability Timeframe) and Biowin 4 (The primary Biodegradation Timeframe) were evaluated together with the Biowin5 (MITI Linear model prediction). These results were again used to obtain a YES or NO Ready Biodegradability Prediction.

### Results:

The Biowin 3 of 1,3-Cyclohexanone and 1,4-Cyclohexanone were both estimated to 2,90 (weeks). The Biowin 4 of 1,3-Cyclohexanone and 1,4-Cyclohexanone were both estimated to be 3,64 (days-weeks) and the Biowin 5 was estimated to be 0,69. Their Ready Biodegradability Prediction was therefore YES (readily biodegradable).

1. Composition comprising a compound of General structure 3, 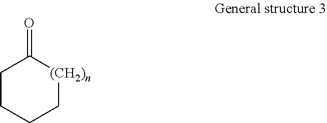
wherein
n is 0-2;
optionally substituted by 1-3 groups selected from ═O, —OH, —C₁₋₆ alkyl, —O—C₁₋₆ alkyl, —CH═O and —C(OH)═O;
or salts, hydrates, solvates or tautomeres thereof,
with the proviso that the compound is not 2-isopropyl-5-methylcyclohexane-1-on;
in an amount preventing biofilm formation.

2. Composition as claimed in claim 1 wherein the composition comprises the compound of General structure 3 in an amount of 0.05-500 mg/ml, preferably 0.1-300 mg/ml or at least in a concentration of 1-100 mg/ml.

3. Composition as claimed in claim 1 wherein the composition is a solution, matrix, powder, gel or coating.

4. Composition as claimed in claim 1
wherein the substituted 1-3 groups are independently selected from the group of carbonyl, —OH, methyl and ethyl,
—O—C₁₋₆ alkyl, —CH═O and —C(OH)═O.

5. Composition as claimed in claim 1
wherein the compound is
substituted by one ═O; and is
optionally substituted by 1-2 groups selected from —OH, —C₁₋₆ alkyl, —O—C₁₋₆ alkyl, —CH═O and —C(OH)═O;
or salts, hydrates, solvates or tautomeres thereof.

6. Composition according to claim 1,
wherein the compound is selected from the group consisting of:
1,3-cyclohexanedione;
1,4-cyclohexanedione;
Cyclopentanone;
5-ethylcyclohexane-1,3 dione and
5-methyl-1,3 cyclohexanedione.

7. Composition as claimed in claim 5 wherein the compound is selected from the group of 1,3-cyclopentadions, a 1,3- or 1,4-cyclohexanedione, and a 1,3- or 1,4-cycloheptadion.

8. Composition as claimed in claim 1, in combination with one or more biocides and/or antibacterial agents.

9. Composition according to claim 8, wherein the biocides and/or antibacterial agent is selected from the group of disinfectants and general biocidal products, preservatives, pest control agents and biocidal products like antifouling agents.

10. Composition according to claim 8 wherein the biocides/or antibacterial agent is selected from the group consisting of 4-hydroxy-3-methoxybenzaldehyde, cetylpyridinium chloride, quorum sensing inhibitors, bioguanides, iodophors, quaternary ammonium compounds, boric acid, cationic tensides, alcohol based, chlorine based, peroxy based and acid based compounds, Tetracyclines, Amphenicols, Beta-lactam antibiotics, Sulphonamides and trimetophrim, macrolides, linkosamides and streptogramins, Aminoglycosides, Quinolones and other antibacterial compounds.

11. Composition according claim 8, wherein the combination with one or more biocides and/or antibacterial agents are for use in a two-step process to prevent biofilm formation.

12. Composition according to claim 1, applicable for either of the following uses:
a. A solvent or solvent mixture;
b. An industrial paint or varnish;
c. Anti-fouling coatings and/or impregnations for marine use;
d. Anti-fouling coatings for maritime use;
e. A coating bound to the surface of or mixed in polymer material;
f. Solutions or coatings attached/linked to inert surfaces;
g. A coating bound to the surface of or mixed in fiber glass materials.

13. Composition according to claim 1 for use in medicine.

14. Composition according to claim 13 for use in a solution, ointment or dressing in human or veterinary medicine or for a medical purpose.

15. Use of a composition comprising one or more of the compounds of General structure 3: 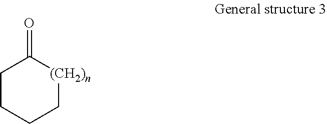
wherein n is 0-2;
substituted by one ═O group; and
optionally substituted by 1-2 groups selected from —OH, —C₁₋₆ alkyl, —O—C₁₋₆ alkyl, —CH═O and —C(OH)═O;
or salts, hydrates, solvates or tautomeres thereof;
in preventing biofilm formation.
